(19) United States
(12) Patent Application Publication
(10) Pub. No.: US 2011/0124056 A1
(43) Pub. Date: May 26, 2011

(54) Pretreatment of Ligno-Cellulosic Biomass with Sulfonation

(75) Inventors: Benjamin Levie, Mercer Island, WA (US); Rajesh Gupta, Katy, TX (US); Johnway Gao, Federal Way, WA (US); Dwight Anderson, Puyallup, WA (US)

(73) Assignee: CHEVRON U.S.A. INC., San Ramon, CA (US)

(21) Appl. No.: 12/894,042

(22) Filed: Sep. 29, 2010

Related U.S. Application Data
(60) Provisional application No. 61/247,444, filed on Sep. 30, 2009.

(51) Int. Cl. C12P 19/04; C12P 19/02; C13K 1/02

(52) U.S. Cl. 435/101; 435/105; 127/37

(57) ABSTRACT

Provided are methods for the pretreatment of ligno-cellulosic biomass such as softwoods with bisulfite such as ammonium bisulfite without the need for exogenous acid. In one variation, a method of pretreating ligno-cellulosic biomass is provided including the following steps: a) providing ligno-cellulosic biomass; b) contacting the ligno-cellulosic biomass with a solution comprising bisulfite at an amount between 1 and 10% of a dry weight of the ligno-cellulosic biomass to form a slurry; c) heating the slurry to a first temperature of 150-210° C. for a first period of time to form a first mixture; d) cooling the first mixture to a second temperature of 100-200° C. to form a second mixture; and e) maintaining the second mixture at the second temperature for a second period of time to form pretreated ligno-cellulosic biomass; wherein the first temperature is higher than the second temperature.

### CROSS-REFERENCE TO RELATED APPLICATION

This application claims priority to U.S. Provisional Application No. 61/247,444, filed on Sep. 30, 2009, the contents of which are incorporated herein by reference in its entirety.

### BACKGROUND

1. Field

The present disclosure relates generally to methods for the pretreatment of ligno-cellulosic biomass with low-acid sulfonation. More specifically it relates to sulfite pretreatment of softwoods that does not require exogenous acid for downstream hydrolysis to sugars.

2. Related Art

Conversion of ligno-cellulosic biomass to biofuels or biomaterials, such as ethanol, mixed alcohols, hydrocarbons, or monomeric or oligomeric sugar intermediates, requires pretreatment of the biomass to make the cellulose accessible to hydrolysis and biologically-based conversion processes. A widespread, potential feedstock is softwood; however, pretreatment of softwood is more challenging than other feedstocks. Softwoods are typically more recalcitrant towards pretreatment and hydrolysis than hardwood, and much more recalcitrant than herbaceous crops, such as corn stover, straw, and switchgrass.

Hydrolysis on softwood can be achieved by acid hydrolysis or by enzymatic hydrolysis after a suitable pretreatment, such as an acid-catalyzed prehydrolysis stage designed to remove a majority of the hemicelluloses. The choice of hydrolysis method depends on a trade-off between yield, cost, severity of pretreatment, and degradation of the biomass to unwanted byproducts. The use of acid in pretreatment, for example, increases the solubility of hemicelluloses during a prehydrolysis step, but does so at the loss of hydrolyzed hemicelluloses and cellulose to byproducts such as acetic acid, hydroxymethylfurfural (HMF), and furfural. The production of HMF and furfural in particular is undesirable both because it represents a yield loss and because the degradation products can interfere with downstream fermentation and processing. Because severe pretreatments are often required for softwood, significant yield losses to degradation products may be observed, thus limiting the amount of hydrolysis that results in carbohydrate monomers and oligomers while necessitating hydrolysate conditioning, such as over-liming, to achieve fermentation. Other approaches have been investigated that can significantly improve hydrolysis efficiency, such as Yang et al. (Yang, B. et al. Biotech. and Bioeng. (2002) 77:678-684). Such treatments illustrate the potential for improving hydrolysis yield, but do not yet offer economically compelling solutions.

One such approach shows the benefit of sulfonating softwood with sodium or magnesium bisulfite. (Zhu, J. Y. et al. Bioresource Technology (2009), 100:2411-2418). Zhu et al. demonstrated that the addition of acid has a beneficial effect on the final yield after enzymatic hydrolysis. They achieved good yields, but the liquor recovery cycle is costly, approaching what would be required for a sulfite mill. The addition of acid also presents a number of disadvantages, including added cost and complexity of an acid addition system and increased byproducts that inhibit downstream processes. Therefore, there is a need for simpler and less costly pretreatment processes that maintain high hydrolysis yields and do not require addition of exogenous acid.

### SUMMARY

The present invention provides methods for the pretreatment of ligno-cellulosic biomass with low-acid sulfonation. More specifically it relates to bisulfite pretreatment of softwoods for downstream hydrolysis to sugars without the need for exogenous acid. The elimination of acid reduces the cost and complexity of the overall process, but high sugar yields are maintained by employing a two-step temperature regime in the pretreatment process.

In one variation, the present invention provides a method of pretreating ligno-cellulosic biomass comprising: a) providing ligno-cellulosic biomass; b) contacting the ligno-cellulosic biomass with a solution comprising bisulfite at an amount between 1 and 10% of a dry weight of the ligno-cellulosic biomass to form a slurry; c) heating the slurry to a first temperature of 150-210° C. for a first period of time to form a first mixture; d) cooling the first mixture to a second temperature of 100-200° C. to form a second mixture; and e) maintaining the second mixture at the second temperature for a second period of time to form pretreated ligno-cellulosic biomass; wherein the first temperature is higher than the second temperature.

In some variations, the first period of time is 1-120 minutes and the second period of time is 30-240 minutes. In other variations, the first period of time is 1-60 minutes and the second period of time is 50-100 minutes.

In some variations, the bisulfite is selected from the group consisting of ammonium bisulfite, sodium bisulfite, calcium bisulfite, and magnesium bisulfite. In some preferred variations, the bisulfite is ammonium bisulfite. In some variations, the amount of bisulfite is between 1 and 10% of the dry weight of the ligno-cellulosic biomass. In other variations, the amount of bisulfite is between 3 and 9% of the dry weight of the ligno-cellulosic biomass.

In some variations, the ligno-cellulosic biomass is selected from the group consisting of softwood, hardwood, switchgrass, corn stover, straw, miscanthus, cane bagasse, recycled paper, waste paper, and agricultural waste. In some preferred variations, the ligno-cellulosic biomass is softwood, hardwood, and switchgrass. In another preferred variation, the ligno-cellulosic biomass is softwood.

In some variations, the first temperature is 140-210° C. and the first period of time is 1-120 minutes. In other variations, the second temperature is 130-180° C. and the second period of time is 30-240 minutes. In other variations, the first temperature is 160-190° C. and the first period of time is 2-30 minutes. In other variations, the second temperature is 130-150° C. and the second period of time is 60-90 minutes.

In some variations, the pretreated ligno-cellulosic biomass has a liquor containing monomeric and/or oligomeric sugars. In other variations, the pretreating forms one or more byproducts selected from the group consisting of hydroxymethylfurfural and furfural, and each byproduct is formed in an amount less than 0.5% of the dry weight of the ligno-cellulosic biomass.

In some variations, the pretreating method further comprises enzymatically hydrolyzing the pretreated ligno-cellulosic biomass. In some variations, the hydrolyzing forms monomeric and/or oligomeric sugars. In other variations, the hydrolyzing forms one or more sugars selected from the group consisting of glucan, xylan, arabinan, mannan, and galactan. In some variations, the sugars are formed in an amount of at least 60% of available sugars in the ligno-cellulosic biomass.

In one preferred variation, the present invention provides a method of pretreating ligno-cellulosic biomass comprising: a) providing ligno-cellulosic biomass; b) contacting the ligno-cellulosic biomass with a solution comprising ammonium bisulfite at an amount between 1 and 10 wt % of a dry weight of the ligno-cellulosic biomass to form a slurry; c) heating the slurry to a first temperature of 150-210° C. for a first period of time of 1-120 minutes to form a first mixture; d) cooling the first mixture to a second temperature of 100-200° C. to form a second mixture; and e) maintaining the second mixture at the second temperature for a second period of time of 60-240 minutes to form pretreated ligno-cellulosic biomass; wherein the first temperature is higher than the second temperature.

In some variations, the pretreated ligno-cellulosic biomass is hydrolyzed to form a hydrolysate. In some variations, the pretreating methods further comprise: f) burning residual remaining in the hydrolysate to produce SO₂; g) scrubbing the SO₂ with ammonia to reform ammonium bisulfite; and h) recycling the reformed ammonium bisulfite for further pretreating.

In some variations, the ligno-cellulosic biomass is prepared by one or more techniques selected from the group consisting of cutting, chipping, grinding, refining, milling, pressing, extruding, crushing, conditioning, cracking, resizing, and screening.

### BRIEF DESCRIPTION OF THE DRAWINGS

FIG. 1. Glucan expressed as a percentage of initial biomass under 3% bisulfite, 1% acid, and 160° C. pretreatment conditions. The quantity of glucan in the hydrolysate was determined after 48 hours of enzymatic hydrolysis of the non-solubilized fraction after pretreatment.

FIG. 2. Degradation byproducts in prehydrolysate expressed as a percentage of initial biomass under 3% bisulfite, 1% acid, and 160° C. pretreatment conditions.

FIG. 3. Glucan expressed as a percentage of initial biomass under 7% bisulfite, 1% acid, and 160° C. pretreatment conditions. The quantity of glucan in the hydrolysate was determined after 48 hours of enzymatic hydrolysis of the non-solubilized fraction after pretreatment.

FIG. 4. Degradation byproducts in prehydrolysate expressed as a percentage of initial biomass under 7% bisulfite, 1% acid, and 160° C. pretreatment conditions.

FIG. 5. Acetic acid formation in one- and two-step temperature regimes under 7% bisulfite pretreatment conditions. Acetic acid concentration under the two-step temperature regime is similar to the low temperature results.

FIG. 6. Hydroxymethylfurfural (HMF) formation in one- and two-step temperature regimes under 7% bisulfite pretreatment conditions. HMF forms quickly at 160° C. The two-step temperature regime has degradation similar to the low temperature case.

FIG. 7. Furfural formation in one- and two-step temperature regimes under 7% bisulfite pretreatment conditions. Furfural forms quickly at 160° C. The two-step temperature regime has degradation similar to the low temperature case.

FIG. 8. Comparison of hydrolysis efficiency with one- and two-step temperature regimes as amount of acid added is varied. Hydrolysis efficiency is the total amount of glucan, xylan, and galactan liberated in the prehydrolysate and the hydrolysate after 48 hours of enzymatic hydrolysis divided by the amount of glucan, xylan, and galactan available in the initial pine harvest residual feedstock. Pretreatment conditions are 7% ammonium bisulfite on wood.

FIG. 9. Comparison of hydrolysis efficiency with one- and two-step temperature regimes as the temperature of the first step of the two-step process is varied. Times at the first temperature were 30 minutes for 160° C., 20 minutes for 170° C., 10 minutes for 180° C., and 2 minutes for 190° C., with the balance of the 90 minutes of total time being at 145° C. Pretreatment conditions are 7% ammonium bisulfite on wood. Hydrolysis yield of two-step temperature regime with or without acid is higher than hydrolysis yield of one-step temperature regime without acid, and comparable to one- or two-step temperature regimes with acid.

FIG. 10. Comparison of hydroxymethylfurfural (HMF) production with one- and two-step temperature regimes as the temperature of the first step of the two-step process is varied as indicated in FIG. 9. Pretreatment conditions are 7% ammonium bisulfite on wood.

FIG. 11. Comparison of furfural production with one- and two-step temperature regimes as the temperature of the first step of the two-step process is varied as indicated in FIG. 9. Pretreatment conditions are 7% ammonium bisulfite on wood.

FIG. 12. Comparison of xylan content as oligomers in the prehydrolysate with one- and two-step temperature regimes as the temperature of the first step of the two-step process is varied as indicated in FIG. 9. Pretreatment conditions are 7% ammonium bisulfite on wood.

FIG. 13. Comparison of galactan content as oligomers in the prehydrolysate with one- and two-step temperature regimes as the temperature of the first step of the two-step process is varied as indicated in FIG. 9. Pretreatment conditions are 7% ammonium bisulfite on wood.

### DETAILED DESCRIPTION

The following description sets forth exemplary methods, parameters and the like. It should be recognized, however, that such description is not intended as a limitation on the scope of the present invention but is instead provided as a description of exemplary embodiments. From these, a person of ordinary skill would be able to practice the invention without undue experimentation.

### 1. DEFINITIONS

As used herein, “pretreatment” refers to the thermo-chemical treatment of biomass in order to make cellulose available to downstream hydrolysis and biologically-based conversion processes.

As used herein, the term “prehydrolysate” refers to the water-soluble fraction of the pretreatment reaction mixture.

As used herein, the term “hydrolysate” refers to the reaction mixture after having undergone enzymatic hydrolysis.

As used herein, “saccharification” refers to the enzymatic hydrolysis of biomass to monomeric and/or oligomeric sugars.

As used herein, “inhibitor(s)” or “byproduct(s)” are used interchangeably, and refer to side products other than sugars that may be present in both the prehydrolysate after pretreatment and the hydrolysate after enzymatic hydrolysis.

### 2. DESCRIPTION OF THE INVENTION

The present invention provides methods for the pretreatment of ligno-cellulosic biomass with low-acid sulfonation. More specifically it relates to bisulfite pretreatment of softwoods for downstream hydrolysis to sugars without the need for exogenous acid. The methods of the present invention employ a two-step temperature regime that allows for the elimination of acid. The elimination of acid reduces the cost and complexity of the overall process, while the two-step temperature regime maintains high sugar yields. A number of process benefits are achieved by eliminating exogenous acid:

1. The process is simpler, avoiding the cost of an acid addition system;

2. The cost of pH control in saccharification is reduced;

3. The production of oligomers is increased, which may be preferred by some processes that would follow the pretreatment process;

4. Depending on the pH range of operation, cost may be reduced because the absence of acid allows for less stringent metallurgy requirements; and

5. The amount of inhibitors formed is reduced, thereby:
i. improving yield by retaining sugars;
ii. avoiding the cost of conditioning such as over-liming;
iii. creating the possibility of not washing the solid fraction, which allows for:
   a. lower capital cost (no washer, or reduced washing);
   b. more favorable energy and water balance; and
   c. improved yield by avoiding dilution/separation losses;
iv. enabling combined pretreated fiber with liquor for direct enzymatic hydrolysis and sequential fermentation (SF);
v. enabling combined pretreated fiber with liquor for simultaneous saccharification and fermentation (SSF); and
vi. enabling combined pretreated fiber with liquor for consolidated bioprocessing (CBP).

Ligno-cellulosic materials treated according to the methods of the present invention generally include softwood, hardwood, switchgrass, corn stover, straw, miscanthus, cane bagasse, recycled paper, waste paper, and agricultural waste. In some preferred variations, the ligno-cellulosic biomass is softwood, hardwood, and switchgrass. Softwood is of particular interest due to its widespread availability and sustainability as a non-food biomass source, although softwood is more recalcitrant towards pretreatment processes than other biomass sources such as grasses, corn stover, or hardwoods. In some variations, the ligno-cellulosic biomass is prepared prior to pretreatment by one or more techniques selected from the group consisting of cutting, chipping, grinding, refining, milling, pressing, extruding, crushing, conditioning, cracking, resizing, and screening.

In some variations, the bisulfite is selected from the group consisting of ammonium bisulfite, sodium bisulfite, calcium bisulfite, and magnesium bisulfite. In one preferred variation, the bisulfite source is ammonium bisulfite. An advantage of using ammonium bisulfite is the relative ease of dealing with the post-pretreatment liquor: it can be burned to produce SO₂, which may be scrubbed with ammonia and used to make-up the ammonium bisulfite liquor. This simplifies the post-pretreatment recovery cycle which lowers the overall cost of the process. In some variations, the amount of bisulfite is between 1 and 10% of the dry weight of the ligno-cellulosic biomass. In other variations, the amount of bisulfite is between 3 and 9% of the dry weight of the ligno-cellulosic biomass.

In one variation, the present invention provides a method of pretreating ligno-cellulosic biomass comprising: a) providing ligno-cellulosic biomass; b) contacting the ligno-cellulosic biomass with a solution comprising bisulfite at an amount between 1 and 10% of a dry weight of the ligno-cellulosic biomass to form a slurry; c) heating the slurry to a first temperature of 150-210° C. for a first period of time to form a first mixture; d) cooling the first mixture to a second temperature of 100-200° C. to form a second mixture; and e) maintaining the second mixture at the second temperature for a second period of time to form pretreated ligno-cellulosic biomass; wherein the first temperature is higher than the second temperature.

In some variations, the first period of time is 1-120 minutes and the second period of time is 30-240 minutes. In other variations, the first period of time is 1-60 minutes and the second period of time is 50-100 minutes.

In some variations, the first temperature is 140-210° C. and the first period of time is 1-120 minutes. In other variations, the second temperature is 130-180° C. and the second period of time is 30-240 minutes. In other variations, the first temperature is 160-190° C. and the first period of time is 2-30 minutes. In other variations, the second temperature is 130-150° C. and the second period of time is 60-90 minutes.

In some variations, the pretreated ligno-cellulosic biomass has a liquor containing monomeric and/or oligomeric sugars. In other variations, the pretreating forms one or more byproducts selected from the group consisting of hydroxymethylfurfural and furfural, and wherein each byproduct is formed in an amount less than 0.5% of the dry weight of the ligno-cellulosic biomass.

In some variations, the pretreating method further comprises enzymatically hydrolyzing the pretreated ligno-cellulosic biomass. In some variations, the hydrolyzing forms monomeric and/or oligomeric sugars. In other variations, the hydrolyzing forms one or more sugars selected from the group consisting of glucan, xylan, arabinan, mannan, and galactan. In some variations, the sugars are formed in an amount of at least 60% of available sugars in the ligno-cellulosic biomass.

In one preferred variation, the present invention provides a method of pretreating ligno-cellulosic biomass comprising: a) providing ligno-cellulosic biomass; b) contacting the ligno-cellulosic biomass with a solution comprising ammonium bisulfite at an amount between 1 and 10 wt % of a dry weight of the ligno-cellulosic biomass to form a slurry; c) heating the slurry to a first temperature of 150-210° C. for a first period of time of 1-120 minutes to form a first mixture; d) cooling the first mixture to a second temperature of 100-200° C. to form a second mixture; and e) maintaining the second mixture at the second temperature for a second period of time of 60-240 minutes to form pretreated ligno-cellulosic biomass; wherein the first temperature is higher than the second temperature.

In some variations, the pretreated ligno-cellulosic biomass is hydrolyzed to form a hydrolysate. In some variations, the pretreating methods further comprise: f) burning residual remaining in the hydrolysate to produce SO₂; g) scrubbing the SO₂ with ammonia to reform ammonium bisulfite; and h) recycling the reformed ammonium bisulfite for further pretreating.

The methods of the present invention may be scaled up to a commercial size by employing existing pulp mill equipment with no or only minor modification, namely to perform cooling if needed between the first and second stage process. The pretreatment process may be carried out in a 2 batch or a single continuous digester, both of which are commonly used at commercial kraft or sulfite pulp mills. A larger average size of biomass will be fed to the commercial digester compared with the laboratory-scale examples discussed below, which were based on a material ground to pass a 10 mesh screen but to be retained on a 20 mesh screen. This was intended to better fit the small size diameter of the lab reactor.

It is noted that unlike a pulp mill, the chip size may be physically reduced without the consequences associated with producing pulp, such as loss in fiber strength. The most significant penalty associated with making the biomass smaller is the energy input required. To this end, biomass in the form of wood chips may be either produced to a smaller average size or may be mechanically altered via low energy devices like hammer mills, chipsizers, crackers, conditioners, screw feeders or slicers. These mechanical processes may change the size of the wood chips and/or destructure the wood to make it more easily penetrable by the pretreatment liquor. Some types of processing may also avoid creating a fines level that could potentially create flow problems in the digester. With this type of equipment, the difference in pretreatment process parameters may be minimized between the fines that are used in the laboratory-scale examples and the more realistically sized biomass in a scaled up plant.

### EXAMPLES

### Feedstock Preparation

Harvest Residuals from pine crop trees in the southeastern United States, a hardwood-rich non-crop understory (referred to as “Early Cleaning”), and unbleached Douglas-fir Thermo-Mechanical Pulp (TMP) were received from Weyerhaeuser Company (Seattle, Wash.). Harvest Residuals, which contained 30% moisture, were sieved and the fraction retained between 10 and 20 mesh was used for doing the pretreatment work. Early Cleaning was received in ground fibrous form with 20% moisture. Poplar chips were received from a third party. All chips were ground further, sieved and the fraction between 10-20 mesh was utilized for pretreatment. TMP was in the pulp form and it was used as it is. All of these feedstocks were stored in refrigerator at less than 4° C.

Switchgrass was received from Mesa Reduction Engineering and Processing (Auburn N.Y.) and had moisture less than 10%. It was stored indoors under ambient conditions. A size between 10-20 mesh was also used for switchgrass.

Reagent Preparation

Sodium bisulfite (Fisher scientific Catalog #AC223075000) was purchased in the powder form and Magnesium bisulfite (Sigma Catalog #398233) was purchased in a form of 30% w/w solution. Ammonium bisulfite was a generous gift from Tessenderlo Kerley, Inc. (Phoenix, Ariz.) in a form of 65% w/w solution. Sulfuric acid (95.8% assay and Catalog #A-300-212) was purchased from Fisher scientific.

These reagents were diluted with deionized water to the appropriate concentration for loading the pre-calculated amount into the reactor. All the reported bisulfite reagents and acid loadings are based on the oven dry (OD) weight of the biomass.

Enzymes Preparation

Cellulase (Celluclast, Sigma Catalog #C-2730) and β-glucosidase (Novozymes-188, Sigma Catalog #C-6105) were primarily used in the enzymatic hydrolysis experiments. The activity of celluclast was found to be approximately 80 FPU/ml as measured in Weyerhaeuser Lab (Seattle, Wash.) and activity of β-glucosidase was reported to be 250 CBU/g (300 CBU/ml) by the supplier (Novozymes). These enzymes were stored in refrigerator below 4° C.

General Pretreatment Method

Hastelloy or stainless steel pencil reactors with 4.25″ (Length) and 0.75″ (Outside diameter) were used for the pretreatment. Reactors were capped from both the sides with reusable Swagelok fittings. 1-2 gram OD biomass was loaded into the reactor with predetermined acid (0-4% of OD biomass weight) and bisulfite solution (3-9% of OD biomass weight). After loading the reactor with biomass and reagents, it was capped and kept left to soak for at least one hour. After the soaking time, the reactor was transferred to a sand bath heater. Before transferring the reactor, the sand bath was preheated to the temperature 20° C. higher than the required reaction temperature. Sand bath temperature was controlled in such a way that heating time to reach the required temperature in the reactor was approximately 5-8 min. Once the reactor attained the required temperature, it was kept there in the sand bath for a predetermined reactor time. After that it was immediately quenched to the very low temperature (<5° C.) by immersing the reactors in the ice cold water. Cooling time was approximately 5 min.

In the pretreatment reactions where the two-step temperature regime was employed and the first temperature is higher than the second temperature, the first temperature was brought down from the higher level (160° C.-210° C.) to the lower level (100° C.-145° C.) by turning down the sand bath heat completely and by increasing the air flow rate to the maximum possible. It took approximately 10-25 min to cool down the reactor from the higher to the lower temperature level depending upon the value of temperature at both levels.

After the pretreatment reaction, the content in the reactor was filtered out by using vacuum filtration. The solid part was washed until it achieved a pH of 7. Filtrate and liquid from washing were combined as pretreatment liquor. Pretreatment liquor was analyzed for sugar (monomer/oligomers) and degradation products. A typical amount of wash water used was 40 to 60 times the dry mass of the sample. Enzymatic hydrolysis of pretreated solids was carried out as per the following procedure.

General Enzymatic Hydrolysis Method

The moisture content of the pretreated biomass is typically in the range of 65-80%. The moisture content in biomass was measured by an infrared moisture balance (Denver Instrument, IR-35). For enzymatic hydrolysis experiments, wet pretreated biomass (as it is) was used, but loading was based upon the oven dry (OD) biomass. All the experiments for enzymatic digestibility of pretreated biomass were done with the loading of 0.2 g OD biomass/10 ml of total reactant volume. The reaction of enzymatic hydrolysis was carried out in 25 ml test tubes with rubber caps and total volume of reactant in that tube was 10 ml. The enzymatic digestibility reaction was carried out according to NREL (National Renewable Energy Laboratory) Laboratory Analytical Procedure by Selig, et al. Selig, M. et al., “Enzymatic Saccharification of Lignocellulosic Biomass”; NREL Technical Report NREL/TP-510-42629; March 2008.

Conditions of the reaction were as follows: 50° C. and pH 4.8. Sodium citrate buffer was used to maintain the pH. Tetracycline and cycloheximide were used as antibiotic as mentioned in the procedure. A magnetic stirrer was used to shake the contents of the test tube. A stirring plate, with test tube rack on it, was kept inside the incubator (Innova 4230, New Brunswick Scientific Co.) to maintain the temperature. Cellulase loading of 12 FPU/g OD biomass and β-glucosidase loading of 18 CBU/g OD biomass were used in all of the enzymatic hydrolysis experiment.

Analytical Procedures

The pretreatment liquor contains sugar extracted from the biomass in the form of monomer as well as oligomer. Liquid analysis for sugars and degradation product was carried out as per NREL Laboratory Analytical Procedure by Sluiter et al. Sluiter, A. et al., “Determination of Sugars, Byproducts, and Degradation Products in Liquid Fraction Process Samples”; NREL Technical Report NREL/TP-510-42623; January 2008.

Composition analysis of the biomass has been done according to the NREL Laboratory Analytical Procedure by Sluiter et. al. Sluiter, A. et al., “Determination of Structural Carbohydrates and Lignin in Biomass”; NREL Technical Report NREL/TP-510-42618; April 2008.

Sugars concentration in liquid after the compositional analysis and enzymatic hydrolysis was determined by HPLC (Agilent Technologies) using a Bio-Rad Aminex HPX-87P. Aminex HPX-87H column was employed to measure the acetic acid, furfural and HMF concentration in liquid.

### Example 1

### Results on Softwoods (Pine Harvest Residuals)

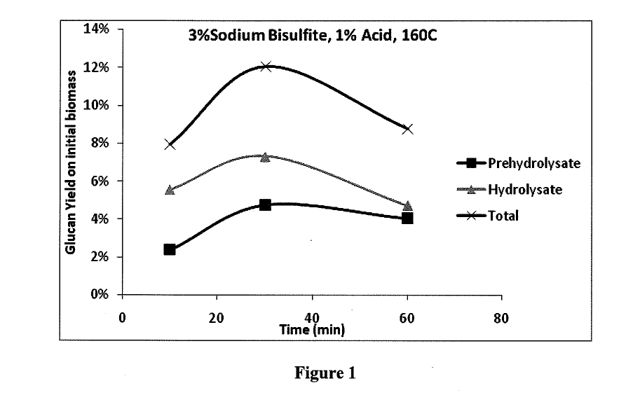

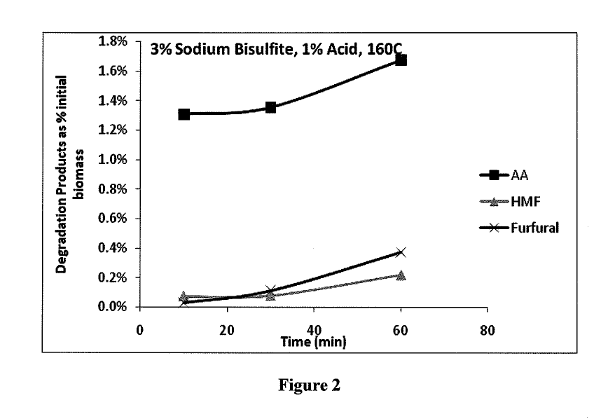

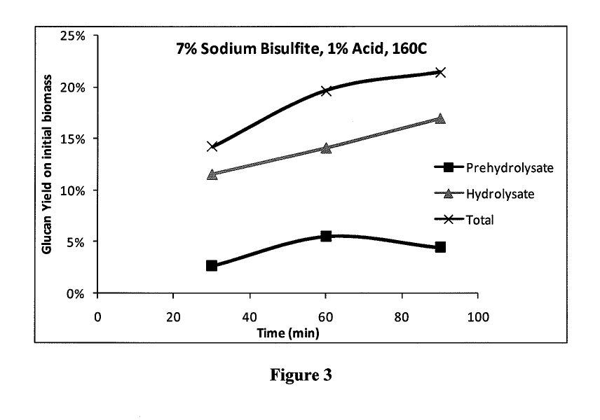

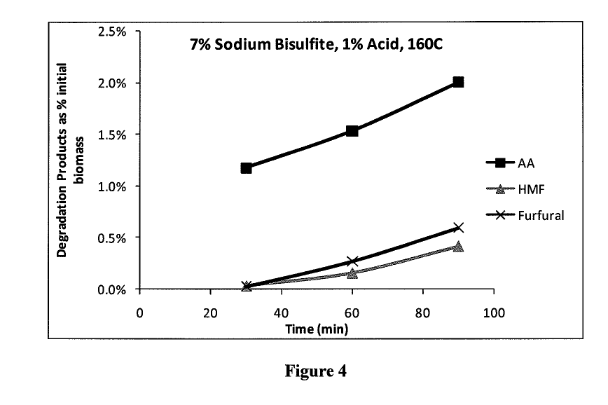

One method of eliminating exogenous acid is to amplify the benefits of sulfonation by controlling the selectivity of degradation reactions compared to the sulfonation reaction. For example, pine harvest residuals were subjected to acid hydrolysis at a 6:1 liquor to wood ratio and 1% acid on wood with the following results at 160° C. As shown in FIG. 1, the solubilized glucan in the prehydrolysate begins to decrease at some point around 30 minutes. This may be due to degradation of glucose and is accompanied by an increase in degradation products, as shown in FIG. 2. FIGS. 3 and 4 show the same effect occurring even at 7% sodium bisulfite concentration. The glucan in the hydrolysate is higher at 7% sodium bisulfite because the sulfonation of the lignin enables enzymes to be used more efficiently, but the rate of formation of degradation products is similar.

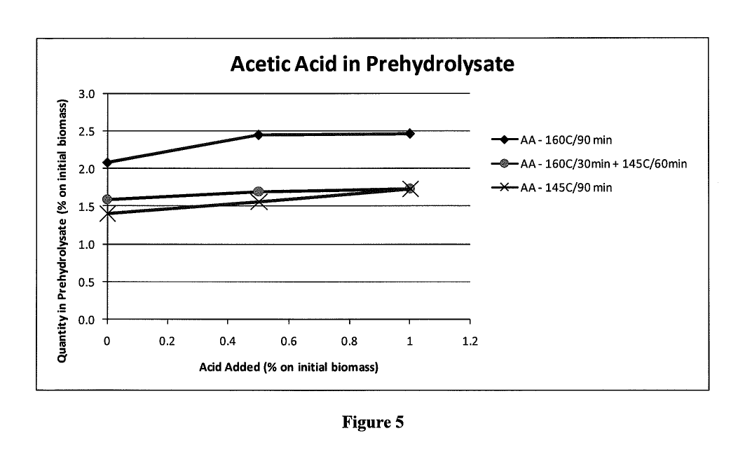

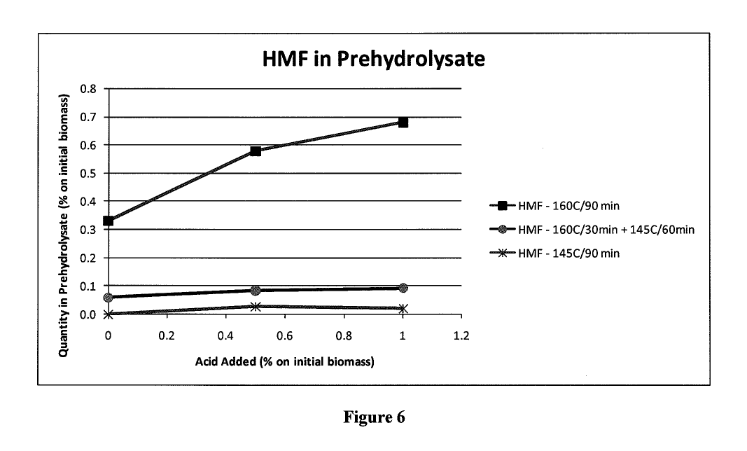

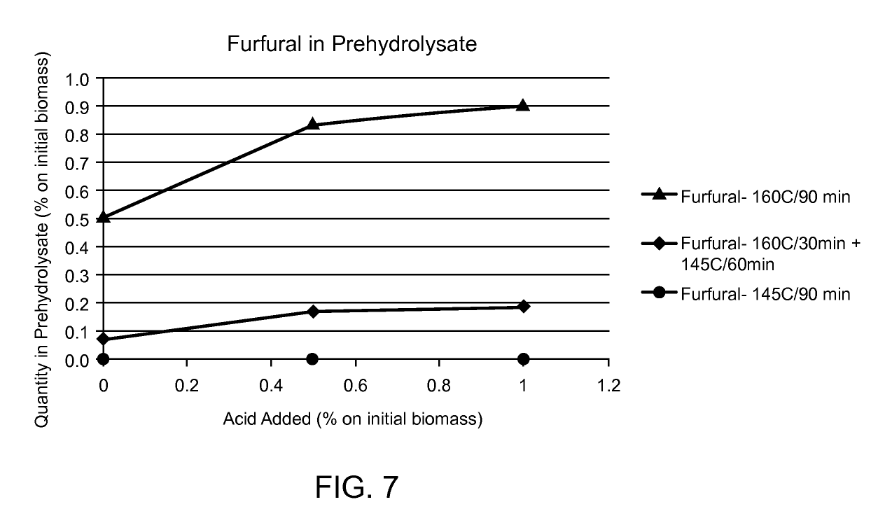

The degradation rates would be lower if acid were decreased. The methods of the present invention provide a two-step temperature regime which allows for the amount of acid to be decreased or eliminated completely. FIGS. 5-7 show the effect of amount of acid added on the amount of byproducts formed in an ammonium bisulfite pretreatment of 7% on biomass. Two of the curves are at a constant temperature of either 145° C. or 160° C. At 145° C., only very low levels of inhibitors are formed. At 160° C., however, the curves for the inhibitors HMF and furfural have a clear positive slope. FIGS. 5-7 also show data from the two-step temperature regime. Like the cases at 145° C. and 160° C., the total time is 90 minutes. In the two-step temperature regime, the first 30 minutes is at 160° C. and the final 60 minutes is at 145° C. The byproduct amount for the two-step temperature regime is very similar to the lower level of the 145° C. runs. The time for the first step was chosen to correspond roughly to the point of inflection in the glucan and xylan content of the prehydrolysate at 30 minutes. In general, an optimal time would decrease as temperature increases, since both solubilization and degradation reactions will be faster.

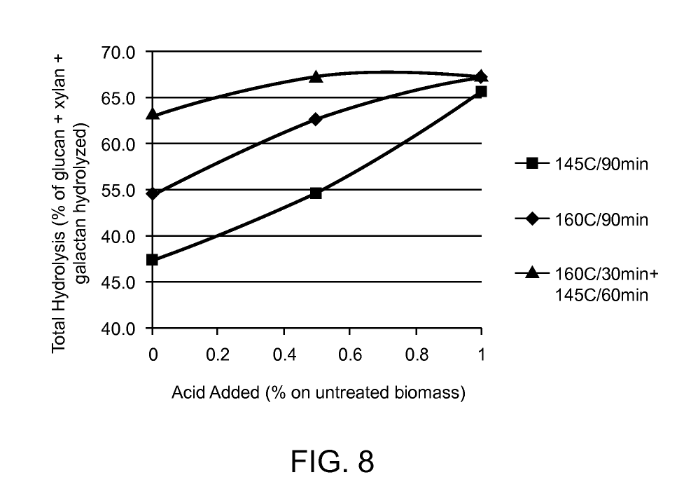

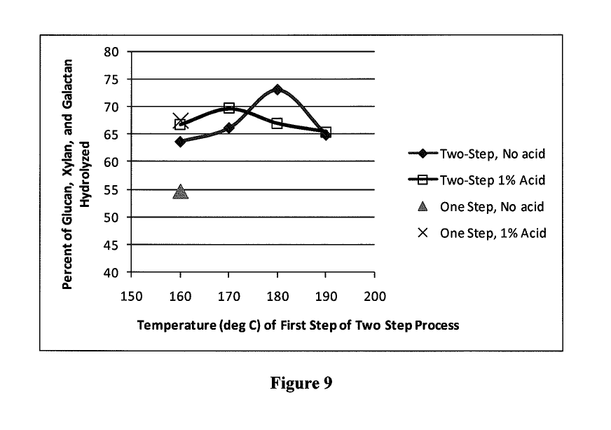

In the methods of the present invention, the two-step temperature regime is effective at hydrolysis with total sugar yields of 60% or greater. FIG. 8 shows that when either acid or temperature is increased, the amount of hydrolysis also increases. What is surprising is that the two-step temperature regime gives results without acid that are comparable to what can be achieved with acid at the higher temperature for the same time (90 minutes). The hydrolysis efficiency was also investigated for two-step temperature regimes as the temperature of the first step of the two-step process is varied as shown in FIG. 9. Times at the first temperature were 30 minutes for 160° C., 20 minutes for 170° C., 10 minutes for 180° C., and 2 minutes for 190° C., with the balance of the 90 minutes of total time being at 145° C. (7% ammonium bisulfite on wood). Hydrolysis yield of two-step temperature regime with or without acid is higher than hydrolysis yield of one-step temperature regime without acid, and comparable to one- or two-step temperature regimes with acid. FIG. 9 also shows that the two-step temperature regime achieves much more similar hydrolysis results over the temperature range from 160° C. to 190° C. than the one-step temperature regime.

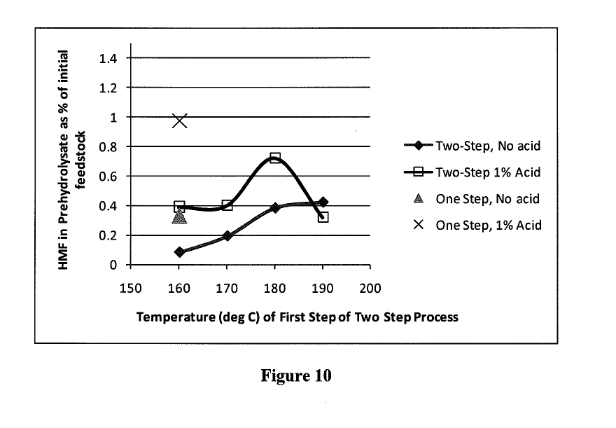

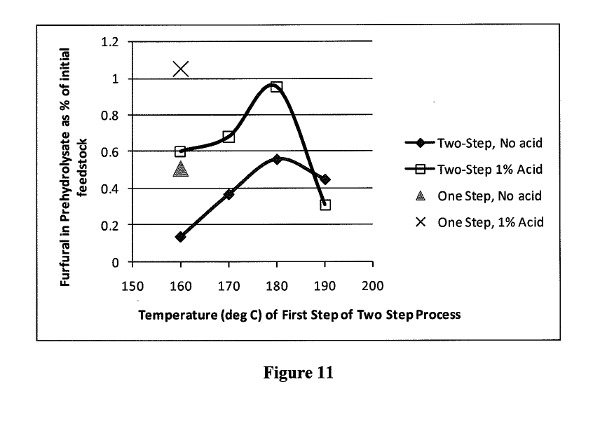

An advantage of the present invention's two-step temperature regime is that the inhibitors formed from the two-step temperature regime without acid are lower than the one-step alternative or even the two-step temperature regime with 1% acid on wood. The differences are greatest at the lower end of the 160° C.-190° C. range. FIGS. 10-11 illustrate this for the byproducts tested. This is significant because these byproducts are also fermentation inhibitors.

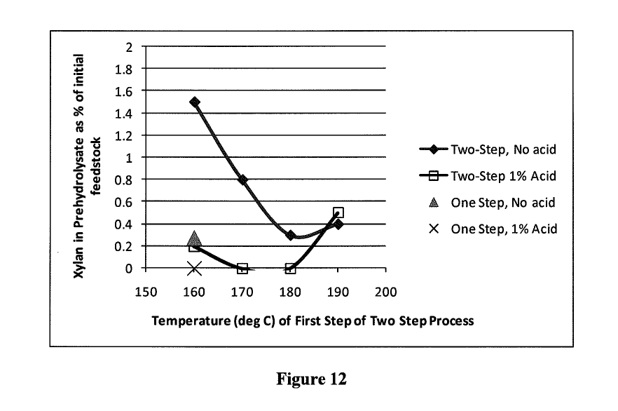

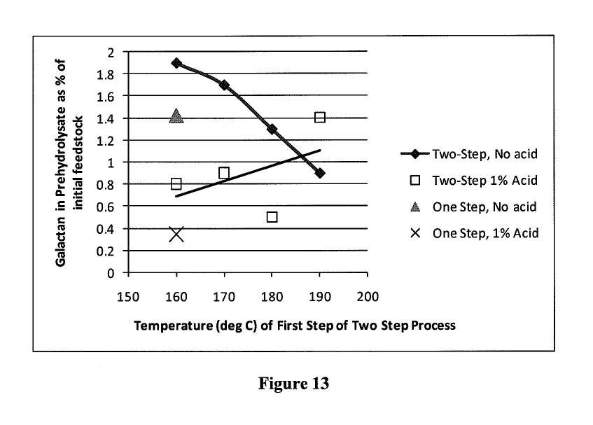

A characteristic of the prehydrolysate that corresponds to the decreased byproducts is the higher oligomeric content of the prehydrolysate. FIGS. 12-13 show that the two-step temperature regime with no acid produces the highest oligomeric content.

### Example 2

### Results on Hardwoods (Early Cleanings and Poplar)

Two hardwood samples were hydrolyzed with this method: poplar and an understory harvest from the southeastern U.S. (“Early Cleanings”) that showed almost entirely hardwood fibers upon fiber analysis. Both furnishes were treated without added acid with 7% ammonium bisulfite on wood for 30 minutes at 160° C. followed immediately by 60 minutes at 145° C. Table 1 shows the results. The non-acid condition for Early Cleanings has an especially good hydrolysis yield of approximately 72%.

TABLE 1
Hardwood examples of the total hydrolysis and the prehydrolysate
content with 7% ammonium bisulfite and two-step temperature
regime 160° C. 30 min./145° C. 60 min. All data expressed as
percentage of initial biomass.

| | % Acid | % Total | % HMF in | % Furfural in |
|---|---|---|---|---|
| Feedstock | Added | Hydrolysis | prehydrolysate | prehydrolysate |
| Early | 0 | 71.7 | 0.0 | 0.1 |
| Cleanings | | | | |
| Early | 1 | 33.4 | 0.0 | 0.0 |
| Cleanings | | | | |
| Poplar | 0 | 63.9 | 0.0 | 0.0 |

### Example 3

### Results on Switchgrass

The same conditions as in Table 1 were run for switchgrass, with both 1% acid on switchgrass and no acid. Preliminary results show solubilization was 5% and 7% for 0% and 1% acid, respectively, and the total amount of hydrolysis was 8% and 23%, respectively. Hydrolysis yields may improve with further optimization.

Although the methods described herein have been described in connection with some variations, it is not intended to be limited to the specific form set forth herein. Rather, the scope of the methods described herein is limited only by the claims. Additionally, although a feature may appear to be described in connection with particular variations, one skilled in the art would recognize that various features of the described variations may be combined in accordance with the methods described herein.

Although individual features of the methods described herein may be included in different claims, these may be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

Terms and phrases used in this document, and variations thereof, unless otherwise expressly stated, should be construed as open ended as opposed to limiting. As examples of the foregoing: the term “including” should be read to mean “including, without limitation” or the like; the terms “example” or “some variations” are used to provide exemplary instances of the item in discussion, not an exhaustive or limiting list thereof; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of methods and compositions described herein may be described or claimed in the singular, the plural is contemplated to be within the scope thereof unless limitation to the singular is explicitly stated. The presence of broadening words and phrases such as “one or more,” “at least,” “but not limited to,” “in some variations” or other like phrases in some instances shall not be read to mean that the narrower case is intended or required in instances where such broadening phrases may be absent.

We claim:

1. A method of pretreating ligno-cellulosic biomass comprising:
a) providing ligno-cellulosic biomass;
b) contacting the ligno-cellulosic biomass with a solution comprising bisulfite at an amount between 1 and 10% of a dry weight of the ligno-cellulosic biomass to form a slurry;
c) heating the slurry to a first temperature of 150-210° C. for a first period of time to form a first mixture;
d) cooling the first mixture to a second temperature of 100-200° C. to form a second mixture; and
e) maintaining the second mixture at the second temperature for a second period of time to form pretreated ligno-cellulosic biomass;
wherein the first temperature is higher than the second temperature.

2. The method of claim 1, wherein the first period of time is 1-120 minutes and the second period of time is 30-240 minutes.

3. The method of claim 1, wherein the first period of time is 1-60 minutes and the second period of time is 50-100 minutes.

4. The method of claim 1, wherein the bisulfite is selected from the group consisting of ammonium bisulfite, sodium bisulfite, calcium bisulfite, and magnesium bisulfite.

5. The method of claim 4, wherein the bisulfite is ammonium bisulfite.

6. The method of claim 1, wherein the ligno-cellulosic biomass is selected from the group consisting of softwood, hardwood, switchgrass, corn stover, straw, miscanthus, cane bagasse, recycled paper, waste paper, and agricultural waste.

7. The method of claim 6, wherein the ligno-cellulosic biomass is selected from the group consisting of softwood, hardwood, and switchgrass.

8. The method of claim 7, wherein the ligno-cellulosic biomass is softwood.

9. The method of claim 1, wherein the amount of bisulfite is between 3 and 9% of the dry weight of the ligno-cellulosic biomass.

10. The method of claim 1, wherein the first temperature is 140-210° C. and the first period of time is 1-120 minutes.

11. The method of claim 1, wherein the second temperature is 130-180° C. and the second period of time is 30-240 minutes.

12. The method of claim 1, wherein the first temperature is 160-190° C. and the first period of time is 2-30 minutes.

13. The method of claim 1, wherein the second temperature is 130-150° C. and the second period of time is 60-90 minutes.

14. The method of claim 1, wherein the pretreated ligno-cellulosic biomass has a liquor containing monomeric and/or oligomeric sugars.

15. The method of claim 1, wherein the pretreating forms one or more byproducts selected from the group consisting of hydroxymethylfurfural and furfural, and wherein each byproduct is formed in an amount less than 0.5% of the dry weight of the ligno-cellulosic biomass.

16. The method of claim 1, wherein the pretreated ligno-cellulosic biomass is hydrolyzed to form a hydrolysate.

17. The method of claim 16 further comprising:
f) burning residual remaining in the hydrolysate after pretreating to produce SO₂;
g) scrubbing the SO₂ with ammonia to reform ammonium bisulfite; and
h) recycling the reformed ammonium bisulfite for further pretreating.

18. The method of claim 1, further comprising enzymatically hydrolyzing the pretreated ligno-cellulosic biomass to form monomeric and/or oligomeric sugars.

19. The method of claim 18, wherein the hydrolyzing forms one or more sugars selected from the group consisting of glucan, xylan, arabinan, mannan, and galactan.

20. The method of claim 19, wherein the sugars are formed in an amount of at least 60% of available sugars in the ligno-cellulosic biomass.
